(19) United States
(12) Patent Application Publication
(10) Pub. No.: US 2025/0057592 A1
(43) Pub. Date: Feb. 20, 2025

(54) ABLATION INSTRUMENT AND MANUFACTURING METHOD THEREOF

(71) Applicant: Erbe Elektromedizin GmbH, Tuebingen (DE)

(72) Inventors: Marcus ADLER, Burladingen (DE); Holger Staebler, Weil im Schoenbuch (DE); Felix Karcher, Tuebingen (DE)

(21) Appl. No.: 18/801,988

(22) Filed: Aug. 13, 2024

(30) Foreign Application Priority Data
Aug. 14, 2023 (EP) 23191383.1

(51) Int. Cl. A61B 18/14; A61B 17/00; A61B 18/00; A61M 25/00

(52) U.S. Cl. CPC A61B 18/1492; A61B 2017/00526; A61B 2018/00077; A61B 2018/00577; A61M 25/001; A61M 25/0045; A61M 25/0068

(57) ABSTRACT

An interior cooled ablation instrument includes a curved section adjoining its distal end in which the ablation instrument is bent relative to its axial direction. The amount of the angle is preferably defined to one-third up to two-thirds, and preferably half of the maximum angulation. The maximum angulation can significantly exceed an absolute value of 140°. Due to the pre-bend in the curved section, a kinking of the hose of the ablation instrument and thus an impairment of the electrode cooling as well as the occurrence of high dam pressure can be effectively avoided. In doing so, the ablation instrument allows a freer work, even more considerably oriented at medical aspects and considerations compared to completely stretched ablation instruments.

### CROSS REFERENCE TO RELATED APPLICATION

This application claims the benefit of European Patent Application No. 23191383.1, filed Aug. 14, 2023, which is incorporated herein by reference in its entirety.

### TECHNICAL FIELD

The invention refers to an ablation instrument, particularly for thermal ablation of a tissue volume by means of current application and in addition to a method for manufacturing such an ablation instrument.

### BACKGROUND

EP 3 788 974 A1 discloses an ablation instrument having a hose-like base body at the distal end of which two ablation electrodes are arranged. The hose is flexible so that the probe can be bent into different directions when inserted into a working channel of an endoscope.

In addition, U.S. Pat. No. 4,474,174 A discloses a surgical instrument for use in a flexible endoscope. This instrument comprises a catheter in which a surgical tool is arranged. This catheter comprises a defined curvature and an opening in the area of the curvature from which a tool guided in the catheter can exit.

U.S. Pat. No. 6,017,340 A discloses another instrument for endoscopic treatment, particularly of the bile duct. This instrument comprises a distal arc-shaped curved end transversed by a wire-shaped electrode.

For carrying out operations at body locations that are difficult to access, U.S. Pat. No. 2,022,065A discloses an instrument having a distal end that is bent backward forming an acute angle.

EP 0 536 440 B1 describes an HF surgical instrument for the endoscopic and/or laparoscopic use inside the rectum, whereby the instrument is curved at the distal end. The defined curvature of the instrument facilitates the guidance of the instrument in a channel of an endoscope, the distal end of which comprises a rigidly bent shape.

Additional prior art is provided by EP 0 947 171 B1 and EP 1 076 523 A1.

### SUMMARY

For the correct operation of an ablation probe it is essential that the electrode being in contact with the tissue does not get too hot in order to avoid a drying of the tissue being in contact with the electrode. To achieve this, the interior lumen of the hose-shaped instrument can be used as cooling channel. Then this cooling channel has to be additionally fluid permeable if the probe is transferred into a bent shape by the endoscope.

It is one object of the invention to provide an ablation instrument, which can be transferred into a bent shape during endoscopic use without limitation of its functionality. Thereby particularly large angulations and tight bending radii shall be made possible. In addition, it is another object of the invention to provide a method for manufacturing of such an ablation instrument.

The ablation instrument according to the invention comprises a flexible hose as base body having at least one lumen that extends from its proximal end up to its distal end. This lumen can be used as cooling channel in order to prevent an electrode arranged on the distal end of the hose from being heated excessively, i.e. to cool the electrode. The electrode can thereby be arranged in a section of the hose that is not curved without application of external forces. In the proximity of the electrode the hose comprises a curved section in which it is configured to follow an arc without external forces acting on the ablation instrument. The distal end section of the hose supporting the electrode and being preferably straight, as well as the section following the arc direction, as well as a section adjoining thereto in proximal direction, the latter being again preferably straight, are configured in a flexible manner. Thereby sections that are not curved in the initial condition can be curved as necessary. In turn, the curved section can be increasingly bent or also transferred into a straight stretched shape. In doing so, the ablation instrument can be inserted into the straight working channel of an endoscope or removed therefrom, even though it comprises a curved section. During this process a curved section is transferred into a non-curved shape.

The curved section can have a stiffness, which is higher than the stiffness of the section adjoining the curved section of the ablation instrument in proximal direction. Also, the stiffness of the curved section can be higher than the stiffness of the section adjoining the curved section in distal direction of the ablation instrument. Alternatively, however, the stiffness of the distal section of the ablation instrument can also be higher than the stiffness of the curved section. In doing so, insertion of the ablation instrument into rigid tissue (for example a solid tumor) can be facilitated.

The curved section is preferably provided in the area of the hose being located in the bent area of the endoscope if the probe is moved with its distal end out of the working channel of the endoscope, so that its electrode or electrodes are inserted into the tissue to be treated. The ablation instrument can also comprise multiple curved sections as necessary.

The curved section preferably defines a curvature angle that is approximately half of the bend to be provided by the ablation instrument. For example, if angulations (i.e. bends) of larger or equal to 145° (for example160°) are desired, the curved section preferably defines an angle to the longitudinal direction of 30° to 70°. Depending on the requirement, also other angles are possible. On one hand, the ablation instrument can be transferred into a stretched shape without being damaged in order to be moved through the working channel of an endoscope and into the tissue of a patient. On the other hand, the curved section of the ablation instrument can be curved due to the respective bent of the distal end of the endoscope up to an angulation angle of 140° to 160°, wherein the curvature of the hose in the curved section increases, however, without the danger that the hose kinks and thus its lumen collapses.

The lumen of the hose is preferably closed at the distal end. Plastic that can be solidified and that forms a plug closing the hose end can serve to close the hose. However, preferably a closure piece is inserted into the hose end that consists at least partly of an electrically conductive material, for example metal. If the closure piece is provided with an individual electrical connection, which can, for example, extend through the lumen of the hose, it can be used as electrode in order to simplify insertion of the instrument into biological tissue and/or to stop bleeding (coagulation). In addition, the closure piece can be connected with a traction means extending from the distal end to the proximal end of the hose in order to avoid damage of the instrument during retraction out of the coagulated tissue.

A capillary channel can be provided inside the hose in order to guide a coolant to the distal end of the ablation instrument in order to receive and dissipate heat originating from the electrode. The capillary channel can be configured inside the hose itself, for example as second lumen. Alternatively, the capillary channel can be configured in a capillary tube arranged in the lumen and extending from the proximal end up to the distal end. The capillary channel is provided with one or more openings from which a coolant can pass into the lumen of the hose in the proximity of the electrode.

Preferably the electrode is configured to enclose the hose and, in a manner, so that it has the same effect in all radial directions. For example, the electrode can be configured as cylinder electrode or by an electrical conductor helically wound around the hose. Thus, the axial orientation of the ablation instrument is irrelevant for the effect on the tissue. If inserted into a stretched straight endoscope, which is bent subsequently, the ablation instrument can therefore, due to a rotation around its axis, take the rotational position in which the curvature of the ablation instrument extends in the same direction as the curvature of the endoscope.

On the distal end of the hose at least one additional electrode can be arranged on a non-curved section. The distal end section of the hose located distally relative to the curved section supports at least two electrodes in this case that, for example, can be used for tissue ablation, particularly for tumor ablation, in that they are connected with two poles of a generator. The latter is preferably configured to create an electrical high frequency voltage in order to allow a current to flow through the tissue being in contact with the two electrodes in order to coagulate the tissue. Basically, however the use of only one electrode on the ablation instrument is possible, wherein a neutral electrode has to be attached to the patient as counter electrode. However, the configuration having two electrodes has advantages, particularly during use in a high ohmic environment, for example during ablation of solid tumors in lung tissue. The electrode or electrodes can be arranged entirely or partly in the curved section of the instrument.

In the ablation instrument according to the invention a coating is arranged on the hose forming a gap or gap-like interstice together with the hose. The coating can extend particularly from the proximal end of the ablation instrument through the curved section up to the electrode or up to an insulator adjoining the electrode. The gap formed between the hose and the coating allows an axial relative movability between the coating and the gap, which supports the flexibility of the ablation instrument.

In the curved section the gap can be filled with a plastic, particularly a two-component plastic, in order to provide a rigid connection between the coating in the hose there. This plastic can be used to define the curvature of the ablation instrument in the curved section. Preferably, the plastic is a flexible plastic, so that the curvature can be eliminated or also increased due to application of external forces. Preferably, the plastic is a plastic that can be solidified, i.e. inserted into the gap during manufacturing of the ablation instrument, whereby concurrently or subsequently the curved section is transferred into its curved shape and the plastic is solidified. Thereby the ablation instrument comprises a section with permanent curvature after solidification of the plastic. This is the case, although the hose as well as the coating are indeed flexible, however are straight, i.e. configured without curvature, in a non-influenced condition. The plastic filling of the gap creates a stiffening of the cross-section, so that the lumen has a reduced sensitivity against collapsing in the area provided with a plastic filling as a result of kinking the ablation instrument.

### BRIEF DESCRIPTION OF THE DRAWINGS

Details of embodiments of the invention as well as potentially also additional aspects thereof are derived from the drawings, the associated description as well as the claims. The drawings show:

FIG. 1 an ablation instrument inserted into an endoscope and connected to a supplying apparatus in a schematic illustration,

FIG. 2 the ablation instrument according to the invention in an illustration in part,

FIG. 3 the distal end of the ablation instrument according to FIG. 2 in enlarged side view,

FIG. 4 the distal end of the ablation instrument according to FIG. 3, but cut longitudinally,

FIG. 5 a part of the curved section of an ablation instrument according to FIGS. 1-4, in schematic longitudinally cut illustration,

FIG. 6 a part of the curved section of an ablation instrument according to FIGS. 1-4, however in a modified embodiment in a schematic longitudinally cut illustration.

### DETAILED DESCRIPTION

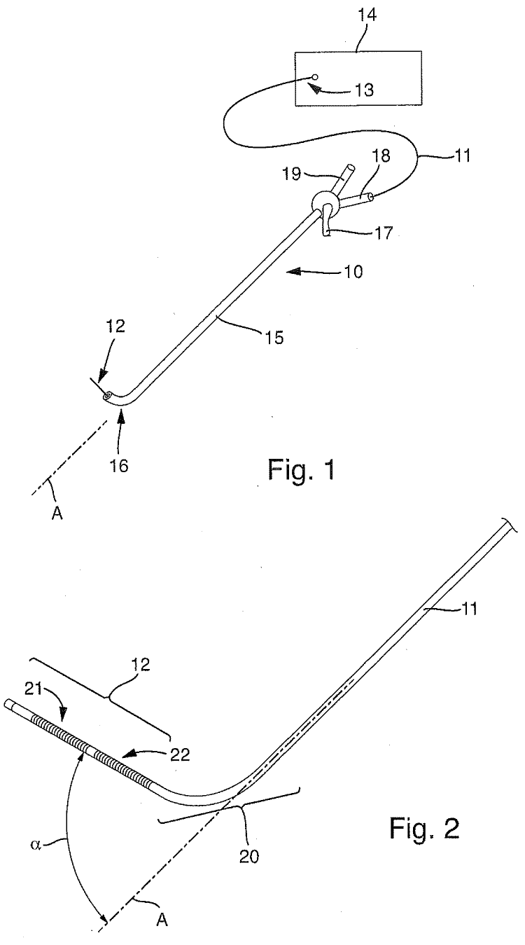

An ablation probe 11 inserted into an endoscope 10 is illustrated in FIG. 1, wherein the distal end 12 of the ablation probe 11 projects from the working channel of the endoscope 10. The proximal end 13 of the ablation probe 11 is, however, connected to a supplying apparatus 14 or an apparatus group that supplies the ablation probe 11 with current, voltage and coolant.

The endoscope 10 comprises a longitudinal straight shank 15 extending along an axial direction A, the distal end 16 of which can be more or less bent by means of an operating element 17, for example an adjusting wheel, an adjusting lever or the like, provided on the proximal end of the shank 15. In the proximity of this operating element 17, endoscope 10 comprises, depending on the number of present working channels, one or more connections 18, 19 via which probes, instruments or the like can be inserted in the respective working channel of the endoscope 10.

Preferably the endoscope 10 is provided for working in tight and angled lumen of a body of a patient. For example, it can be used for treating lung tissue, particularly tumors in lung tissue. The distal end 16 of endoscope 10 can be brought into a straight shape for this purpose by means of a respective actuation of the operating element 17 or also in order to be bent about an angle of, for example, up to 140° or more (150°, 160°) relative to the axial direction A. Thereby bending radii can be created that are smaller than 15 mm.

FIG. 2 illustrates the ablation probe 11 individually based on its distal end 12 and the section adjoining proximally thereto. This section comprises a curved section 20 directly adjoining the distal end 12 in which the ablation probe 11 is curved without influence of external forces. On the contrary, the distal end 12 as well as the section starting at the curved section 20 and extending up to the proximal end 13 of the probe are preferably straight without influence of external forces. This straight section of the ablation probe 11 starting from the curved section 20 and extending into the proximal direction defines a longitudinal direction, which coincides with the axial direction A of the working channel of shank 15 if ablation instrument 11 is inserted into the working channel. The angle α defined by curved section 20 between the distal end 12 and the axial direction A is preferably half of the maximum angulation that has to be achieved by the distal end 16 of shaft 15. For example, if a maximum angulation or bending relative to the axial direction A of 150° shall be achieved, the angle α can be preferably between 30° and 70°, preferably approximately 50° (+/−20°). In doing so, the required deformation of the ablation instrument 11 out of the initial shape into the maximum stretched shape or out of the initial shape into the maximum bent shape is only so small respectively that a kinking of the ablation probe at the bending location is nothing to worry about.

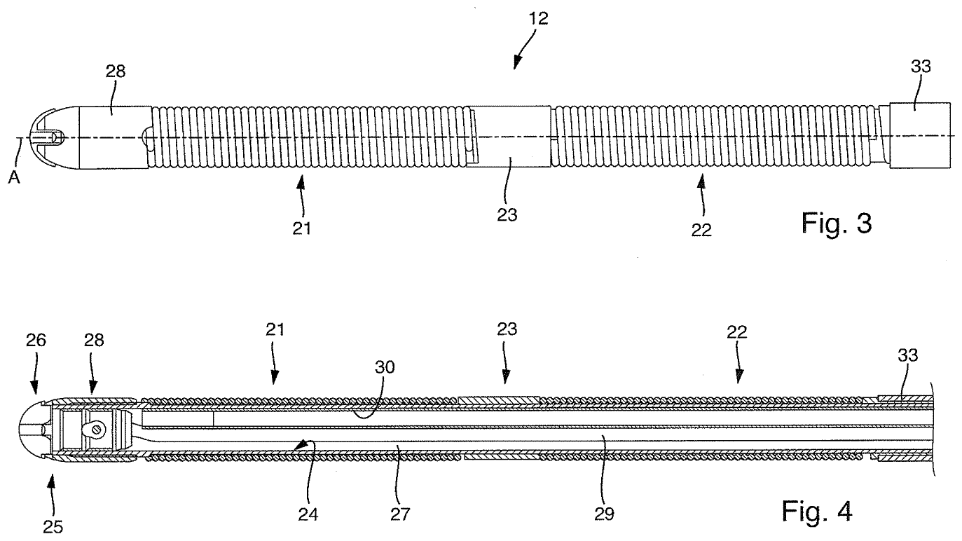

The ablation probe 11 comprises at its distal end 12 at least one, preferably two electrodes 21, 22 that can be rigid or flexible. The electrodes 21, 22 can be configured identically compared with one another. They have identical electrical activities in each radial direction. In other words, they extend around the entire circumferential direction of ablation probe 11. As particularly apparent from FIG. 3, the electrodes 21, 22 can be formed by a helically wound electrical conductor. An insulator 23 can be provided between electrodes 21, 22.

As can be seen in FIG. 4, the two electrodes 21, 22, as preferably also the sleeve-shaped insulator 23, can be arranged on a hose 24 that extends from the proximal end 13 over the entire length of the ablation instrument 11 through the electrodes 21, 22 up to the instrument tip 25. The instrument tip 25 is formed by an end piece 26 with which the lumen 27 surrounded by hose 24 is closed. For example, end piece 26 can consist of metal and can comprise a cross-shaped tip. An insulator element 28 can cover the end piece 26 partly and can extend over the hose 24. The end piece 26 can be connected with the proximal end of the ablation instrument 11 or with a section of the instrument remaining outside of the endoscope during endoscopic use of the ablation instrument by a traction means 29, for example in form of a wire or the like. In addition, in the lumen 27 a fluid line can be arranged that guides a coolant from the proximal end of the ablation instrument 11 up to the distal end 12 in order to cool electrodes 21, 22 therewith. The fluid line can comprise a capillary tube 30, particularly in the distal area, which can also serve for traction force transmission. The capillary tube 30 can comprise one or more coolant exit openings in the area of the electrodes 21, 22, for example at its distal end or in its wall, in order to achieve a cooling effect there.

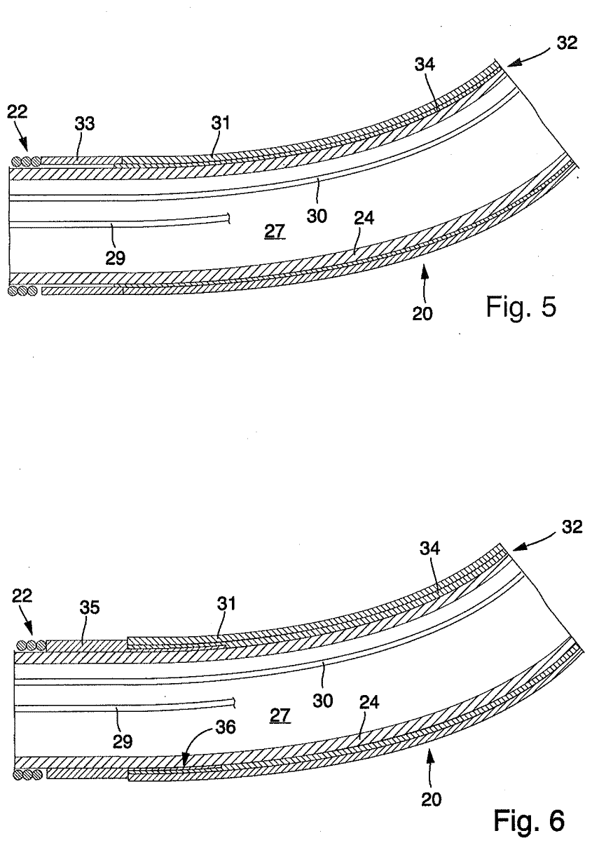

As shown by FIG. 4 and particularly FIG. 5, hose 24 is at least apart from its distal end 12 surrounded by a coating 31, the inner diameter of which exceeds the outer diameter of hose 24 slightly, so that a gap 32 is defined between hose 24 and coating 31. The coating 31 can thereby extend from the proximal end 13 up to one of the electrodes 21, 22. Optionally an insulator ring 33 can be arranged between the distal end of the coating 31 and the electrode 22 adjoining the electrode 22.

A particularity of the ablation instrument 11 according to the invention is the curved section 20 in which the gap 32 is filled by a plastic 34. Preferably, the plastic filling extends, however, not along the entire length of the ablation instrument 11, but is substantially limited to the curved section 20. However, it is also possible to fill larger areas of gap 32 or the entire gap 32 with plastic 34.

Instead of the insulator ring 33, also a ring 35 of metal or plastic can be provided, which particularly serves as centering element and is apparent from FIG. 6. The ring 35 comprises a tube-shaped extension 36 extending into the gap 32 and centers coating 31 relative to hose 24. The plastic 34 filling the gap 32 in the curved section 20 can extend and seal between coating 31 and extension 36.

The plastic 34 is preferably a curable plastic, for example a two-component plastic, which is inserted during the manufacturing of the instrument in liquid form into the gap 32, particularly into the curved section 20. For forming the curved section 20, the latter is transferred into a curved shape shortly before or shortly after insertion of the plastic, whereafter the plastic 34 is cured. For this purpose, ablation instrument 11 can be placed into a respective holding device that forces the ablation instrument 11 in the curved section 20 into an arc shape. After solidification of the plastic the ablation instrument 11 can be removed from the curved form, whereby the area of the ablation instrument 11 that has been held in the curved shape during solidification of the plastic 34 forms the curved section 20. However, the remaining sections of the ablation instrument 11 are provided in stretched form.

The ablation instrument 11 described so far operates as follows:

For use on the patient the endoscope 10 is first inserted into the patient, for example into the bronchial system of the patient and placed at the site to be treated. Already prior to this or now at the latest, the ablation instrument 11 is moved via the connection 18 through the working channel of the endoscope 10 to the operation site. Thereby the pre-bent ablation instrument 11 according to FIG. 2 is transferred elastically into its stretched form and is forwarded up to the bent end 16 of shank 15. As soon as the curved section 20 of ablation instrument 11 reaches the bent distal end 16 of shank 15, it carries out an axial rotation movement due to its tendency to snap back into its pre-curved shape (at least if it does not already have the correct rotational orientation around its axis A by accident). Concurrently, with the axial forward movement of ablation instrument 11 it can penetrate or can be inserted with its probe tip, i.e. with its end piece 26, into the tissue to be treated. For this purpose, the end piece 26 can also be applied with voltage and current where appropriate in order to simplify inserting into more compact tissue, for example tumor tissue or in order to coagulate minor bleedings. The current supplied to the end piece 26 can be realized via traction means 29 or also via capillary tube 30 or an individual conductor.

Due to the pre-bend provided by the curved section 20, the distal end 16 of shank 15 can be bent by a very small bending radius of, for example, less than 15 mm also to large obtuse angles relative to the axial direction A of, for example, more than 145° without having to vary a kinking of hose 24 and thus a collapsing of lumen 27. In addition, the solidified adhesive or plastic 34 as well as cover 31 have a supporting effect and avoid in addition any kinking tendency of hose 24. As a result, coolant supplied via capillary tube 30 can freely flow through lumen 27 back to apparatus 14 or to an outlet provided in the proximity of proximal end 13 without being dammed and thus without lowering the cooling effect on the electrodes 21, 22 or without initiating burst also in a sharp angled condition of ablation instrument 11. The instrument according to the invention allows, in this manner, also treatment of tumors or other tissue parts, which are otherwise difficult to access, which can only be reached by a sharp angulation of end 16 of endoscope 10.

The interior cooled ablation instrument 11 according to the invention comprises adjoining to its distal end 12 a curved section 20 in which the ablation instrument 11 is bent relative to its axial direction A. The amount of this angle is preferably defined to one third up to two thirds, preferably half of the maximum angulation. The maximum angulation can significantly exceed an absolute value of 140°. Due to the pre-bend in the curved section 20, a kinking of the hose of the ablation instrument and thus an impairment of the electrode cooling as well as the occurrence of high dam pressure can be effectively avoided. In doing so, the ablation instrument 11, according to the invention, allows a freer work, even more considerably orientated at medical aspects and considerations compared to completely stretched ablation instruments. The treatment safety for the patient is remarkably increased.

### LIST OF REFERENCE SIGNS

10 endoscope
11 ablation probe
12 distal end of ablation probe 11
13 proximal end of ablation probe 11
14 apparatus for operation of ablation probe 11
15 shank of endoscope 10
16 distal end of shank 15
17 operating element of endoscope 10
18, 19 connections of endoscope 10
A axial direction
20 curved area of ablation probe 11
α angle
21, 22 electrodes
23 insulator
24 hose
25 instrument tip
26 end piece
27 lumen
28 insulator
29 traction means
30 capillary tube
31 coating
32 gap between coating 31 and hose 24
33 insulator
34 plastic
35 ring
36 extension

1. An ablation instrument (11), comprising:
a flexible hose (24) comprising at least one lumen (27) that extends from a proximal end (13) of the hose (24) to a distal end (12) of the hose (24);
at least one electrode (21, 22) that is arranged on the distal end (12) of the hose (24); and
wherein the ablation instrument (11) comprises a curved section (20) in which the hose (24) is configured to follow an arc without external forces applied to the ablation instrument (11).

2. The ablation instrument according to claim 1, wherein the at least one lumen (27) is closed at a tip of the distal end (12).

3. The ablation instrument according to claim 2, further comprising a closure piece (26) for closing the hose (24) that is connected with a traction means (29) extending from the distal end (12) in a proximal direction.

4. The ablation instrument according to claim 1, wherein a capillary channel is disposed inside the hose (24) that comprises one or more openings proximal to the at least one electrode (21).

5. The ablation instrument according to claim 4, wherein the capillary channel is formed in a capillary tube (30) extending through the at least one lumen.

6. The ablation instrument according to claim 1, wherein the at least one electrode (21) is configured to surround the hose (24), wherein the at least one electrode (21) is formed by an electrical conductor wound helically around the hose (24).

7. The ablation instrument according to claim 1, wherein the at least one electrode (22) is positioned outside of the curved section (20).

8. The ablation instrument according to claim 1, wherein at least one additional electrode (22) is arranged on the hose (24) at the distal end (12) at a non-curved section thereof.

9. The ablation instrument according to claim 8, the wherein the at least one electrode and the at least one additional electrode (21, 22) are spaced apart from one another.

10. The ablation instrument according to claim 1, further comprising a coating (31) disposed on the hose (24).

11. The ablation instrument according to claim 10, that wherein a gap (32) is formed between the coating (31) and the hose (24).

12. The ablation instrument according to claim 11, wherein at least one electrical conductor is disposed inside the gap (32) at least one electrical conductor is arranged that extends from the electrode (21) in a proximal direction.

13. The ablation instrument according to claim 11, wherein the gap (32) is filled with a plastic (34) at least in the curved section (20).

14. The ablation instrument according to claim 13, wherein the plastic (34) is a flexible plastic.

15. A method for manufacturing an ablation instrument according to claim 13, comprising:
inserting the plastic (34) into the gap (32), wherein the plastic (34) is a curable plastic;
bending a portion of the ablation instrument (11) into an arc shape to form the curved section; and
curing the curable plastic (34).
